(19) United States
(12) Patent Application Publication
(10) Pub. No.: US 2025/0359868 A1
(43) Pub. Date: Nov. 27, 2025

(54) ANVIL BUTTRESS ATTACHMENT FOR SURGICAL STAPLING APPARATUS

(71) Applicant: Covidien LP, Mansfield, MA (US)

(72) Inventor: Ellianna K. BOOTHE, North Haven, CT (US)

(21) Appl. No.: 18/872,154

(22) PCT Filed: Jun. 26, 2023
(86) PCT No.: PCT/IB2023/056570

§ 371 Date: Dec. 5, 2024

Related U.S. Application Data
(60) Provisional application No. 63/355,907, filed on Jun. 27, 2022.

(51) Int. Cl. A61B 17/072; A61B 17/00

(52) U.S. Cl. CPC A61B 17/072; A61B 2017/00477; A61B 2017/0053; A61B 2017/07257; A61B 2017/07271

(57) ABSTRACT

An anvil buttress loading system includes an anvil assembly having a snap clip disposed on a tissue facing surface, a loading tool including a clip roller extending proximally from a support plate, and an anvil buttress including a buttress body. The snap clip is movable between closed and open positions having generally concave and convex shapes, respectively, relative to the tissue facing surface. The anvil buttress is releasably retained on the loading tool and is transferrable from the loading tool to the anvil assembly such that the buttress body is captured between the snap clip and the tissue facing surface of the anvil assembly. The snap clip is movable from the closed position to the open position by the clip roller when advancing the loading tool onto the anvil assembly and from the open position to the closed position when separating the loading tool from the anvil assembly.

### FIELD

This disclosure relates generally to surgical devices, and more particularly, to anvil buttress attachment systems, assemblies, and methods for releasably securing an anvil buttress to a surgical stapling apparatus.

### BACKGROUND

Surgical stapling apparatus are employed by surgeons to sequentially or simultaneously apply one or more rows of fasteners, e.g., staples or two-part fasteners, to body tissue for the purpose of joining segments of body tissue together. Such apparatus generally include a pair of jaws or finger-like structures between which the body tissue to be joined is placed. When the surgical stapling apparatus is actuated, or “fired”, longitudinally moving firing bars contact staple drive members in one of the jaws. The staple drive members push the surgical staples through the body tissue and into an anvil in the opposite jaw which forms the staples. If body tissue is to be removed or separated, a knife blade can be provided in the jaws of the apparatus to cut the body tissue between the lines of staples.

Surgical supports, e.g., meshes or buttress materials, may be used in combination with surgical stapling apparatus to bridge, repair, and/or reinforce tissue defects within a patient. A clinician may manually attach buttress material(s) to the surgical stapling apparatus in the operating room during a surgical procedure, or utilize a surgical stapling apparatus including the buttress material(s) pre-installed thereon, e.g., by an expensive automated attachment process. The buttress material reinforces the staple or suture line as well as covers the juncture of the tissues to reduce leakage prior to healing.

### SUMMARY

This disclosure relates to anvil side buttress material attachment onto a jaw assembly of a surgical stapling apparatus. Anvil buttress attachment systems, assemblies, and methods of this disclosure are designed to provide robust, releasable mechanical attachment of an anvil buttress to an anvil assembly, and to make anvil side buttress material attachment in the operating room a simple, straightforward, and cost-effective procedure.

In one aspect, this disclosure provides an anvil buttress loading system including an anvil assembly, an anvil buttress loading tool, and an anvil buttress. The anvil assembly includes a tissue facing surface and a snap clip disposed on the tissue facing surface. The snap clip is movable between a closed position having a generally concave shape relative to the tissue facing surface and an open position having a generally convex shape relative to the tissue facing surface. The anvil buttress loading tool includes a carrier having a support plate and a clip roller extending proximally from the support plate. The anvil buttress includes a buttress body. The anvil buttress is releasably retained on the anvil buttress loading tool with the buttress body positioned on the support plate. The anvil buttress is transferrable from the anvil buttress loading tool to the anvil assembly such that the buttress body is captured between the snap clip and the tissue facing surface of the anvil assembly. The snap clip is movable from the closed position to the open position by the clip roller when advancing the anvil buttress loading tool onto the anvil assembly and from the open position to the closed position when separating the anvil buttress loading tool from the anvil assembly.

The clip roller may be configured to move a free end of the snap clip off of the tissue facing surface of the anvil assembly when the anvil buttress loading tool is advanced onto the anvil assembly and to move the free end of the snap clip towards the tissue facing surface when the anvil buttress loading tool is separated from the anvil assembly. The clip roller may include a post extending proximally from the support plate and a cylinder extending laterally from the post.

In the closed position, a free end of the snap clip may be adjacent to the tissue facing surface and, in the open position, the free end of the snap clip may be spaced from the tissue facing surface. The snap clip may include a proximal end secured to the tissue facing surface of the anvil plate and a distal end movable relative to the tissue facing surface. The distal end may be configured to hold the anvil buttress against the tissue facing surface of the anvil assembly.

The snap clip may have a generally tear drop shape and define an opening therethrough. The snap clip may include a proximal base and a distal tip, the proximal base defining the largest width dimension of the snap clip and the distal tip defining the smallest width dimension of the snap clip. A pair of arms may extend from opposed sides of the proximal base. The pair of arms may converge towards one another and connect at the distal tip.

The snap clip may be disposed on a proximal portion of the anvil assembly and be configured to retain a proximal portion of the anvil buttress against the tissue facing surface of the anvil assembly. The anvil assembly may include an anvil tip and the anvil buttress may include a distal window defined through a distal portion of the anvil buttress. The distal window may be configured to engage the anvil tip to retain the distal portion of the anvil buttress on the anvil assembly.

In another aspect, this disclosure provides an anvil buttress loading assembly including an anvil buttress loading tool and an anvil buttress. The anvil buttress loading tool includes a carrier having a support plate and a clip roller extending proximally from the support plate. The anvil buttress includes a buttress body. The anvil buttress is releasably retained on the anvil buttress loading tool with the buttress body positioned on the support plate.

The clip roller may include a post extending proximally from the support plate and a cylinder extending laterally from the post.

The anvil buttress loading tool may include a pair of proximal protrusions extending from the support plate and the anvil buttress may include a pair of proximal tabs extending from the buttress body. The pair of proximal tabs may be engaged with the pair of proximal protrusions. The clip roller may be disposed between the pair of proximal protrusions.

The anvil buttress loading tool may include a cover disposed over the support plate of the carrier. The cover may include a distal protrusion extending outwardly from an outer surface of the cover and a distal opening disposed proximal to the distal protrusion. The anvil buttress may include a distal flap, and the distal flap may extend through the distal opening and be engaged with the distal protrusion.

In yet another aspect, this disclosure provides a tool assembly including a staple cartridge assembly and an anvil assembly. The anvil assembly includes a tissue facing surface and a snap clip disposed on the tissue facing surface. The snap clip is movable between a closed position having a generally concave shape relative to the tissue facing surface and an open position having a generally convex shape relative to the tissue facing surface.

In the closed position, a free end of the snap clip may be adjacent to the tissue facing surface and, in the open position, the free end of the snap clip may be spaced from the tissue facing surface.

The tool assembly may include an anvil buttress having a proximal portion and a distal portion. The proximal portion may be releasably retained on the anvil assembly by the snap clip when the snap clip is in the closed position. The anvil assembly may include an anvil tip and the distal portion of the anvil buttress may define a distal window therethrough. The distal portion may be releasably retained on the anvil assembly by engagement of the anvil tip through the distal window.

The details of one or more aspects of the disclosure are set forth in the accompanying drawings and the description below. Other aspects, as well as features, objects, and advantages of the aspects described in this disclosure will be apparent from the description and drawings, and from the claims.

### BRIEF DESCRIPTION OF DRAWINGS

FIG. 1 is a perspective view of a surgical stapling apparatus including a tool assembly in accordance with an aspect of the disclosure;

FIG. 2 is a bottom, perspective view of an anvil assembly of the tool assembly of FIG. 1;

FIG. 3 is a close-up view of the area of detail indicated in FIG. 2, illustrating a snap clip of the anvil assembly;

FIG. 4 is a perspective view, with parts separated, of an anvil buttress loading tool and an anvil buttress in accordance with aspects of the disclosure;

FIG. 5 is a close-up view of the area of detail indicated in FIG. 4, illustrating a clip roller of the anvil buttress loading tool;

FIG. 6 is a bottom, perspective view of the anvil buttress loading tool of FIG. 5, shown loaded with the anvil buttress of FIG. 5;

FIG. 7 is a perspective view of the anvil assembly of FIG. 2 and the loaded anvil buttress loading tool of FIG. 6, shown with the loaded anvil buttress loading tool partially advanced onto the anvil assembly prior to the clip roller of the anvil buttress loading tool contacting the snap clip of the anvil assembly, the snap clip is disposed in a closed position;

FIG. 8 is a close-up view of the area of detail indicated in FIG. 7;

FIG. 9 is a cross-sectional view of the anvil assembly and the loaded anvil buttress loading tool of FIG. 7, taken along section line 9-9 of FIG. 8;

FIG. 10 is a cross-sectional view of the anvil assembly and the loaded anvil buttress loading tool of FIG. 9, shown with the clip roller of the anvil buttress loading tool engaged with the snap clip of the anvil assembly and transitioning the snap clip from the closed position to an open position during advancement of the anvil buttress loading tool onto the anvil assembly;

FIG. 11 is a cross-sectional view of the anvil assembly and the loaded anvil buttress loading tool of FIG. 10, shown with the anvil buttress loading tool fully advanced onto the anvil assembly and the snap clip disposed in the open position;

FIG. 12 is a cross-sectional view of the anvil assembly and the loaded anvil buttress loading tool of FIG. 11, shown with the anvil buttress loading tool partially retracted off of the anvil assembly and with the clip roller of the anvil buttress loading tool engaged with the snap clip of the anvil assembly and transitioning the snap clip from the open position the closed position;

FIG. 13 is a bottom, perspective view of the anvil assembly of FIG. 12 loaded with the anvil buttress after the anvil buttress loading tool is separated from the anvil assembly; and

FIG. 14 is a close-up view of the area of detail indicated in FIG. 13, illustrating a proximal portion of the anvil buttress secured to the anvil assembly by the snap clip.

### DETAILED DESCRIPTION

Embodiments of this disclosure will now be described in detail with reference to the drawing figures wherein like reference numerals identify similar or identical elements. Throughout this description, the term “proximal” refers to a portion of a structure, or component thereof, that is closer to a user, and the term “distal” refers to a portion of the structure, or component thereof, that is farther from the user.

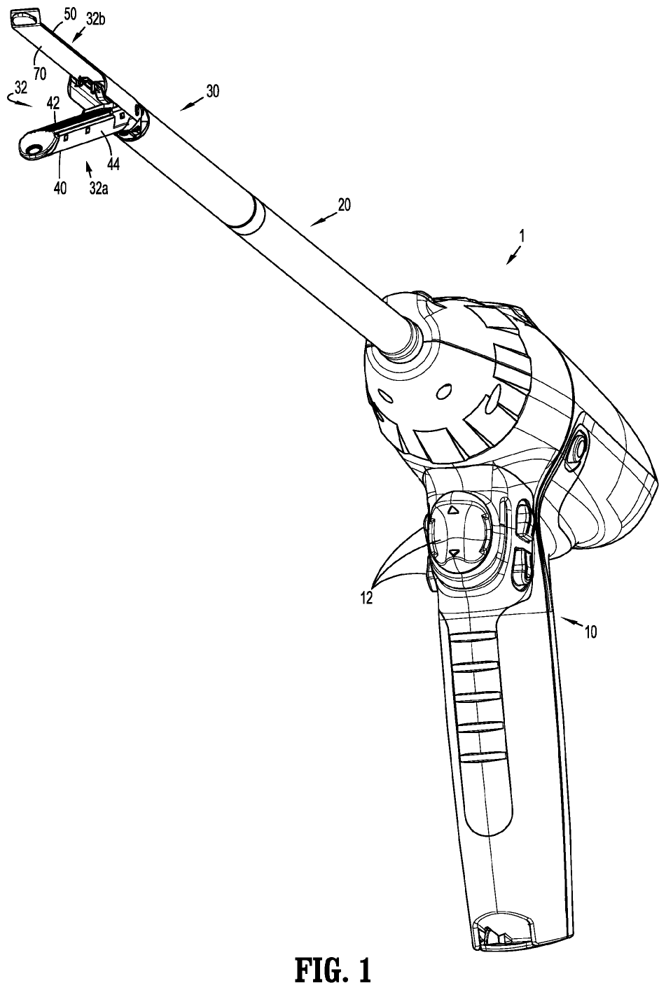

Referring now to FIG. 1, an exemplary surgical stapling apparatus or surgical stapler 1 is shown for use in stapling tissue in accordance with aspects of this disclosure. The surgical stapling apparatus 1 will be described to the extent necessary to disclose aspects of the disclosure. The surgical stapling apparatus 1 generally includes a handle assembly 10, an elongate tubular body 20 extending distally from the handle assembly 10, and a loading unit 30 extending distally from the elongate tubular body 20. The surgical stapling apparatus 1 may be manually driven, partially powered, or fully powered (e.g., by an electric motor) and includes actuators 12 (e.g., finger-actuated control buttons, knobs, toggles, slides, interfaces, and the like) for activating various functions of the surgical stapling apparatus 1.

For a detailed description of the structure and function of exemplary surgical stapling apparatus suitable for use with aspects of this disclosure, including exemplar manual and powered surgical devices, reference may be made to U.S. Pat. Nos. 5,762,256, 5,865,361, and 10,426,468, the entire contents of each of which are incorporated herein by reference. It should be appreciated that principles of this disclosure are equally applicable to surgical stapling apparatus having other configurations such as, for example, the types described in U.S. Pat. Nos. 5,810,240 and 7,334,717, the entire contents of each of which are incorporated herein by reference. Accordingly, it should be understood that a variety of surgical stapling apparatus may be utilized with aspects of the disclosure. For example, laparoscopic or open staplers, such as, for example, GIA™, Endo GIA™, TA™, and Endo TA™ staplers and/or linear and radial reloads with, for example, Tri-Staple™ technology, available through Medtronic (North Haven, CT) may be utilized with aspects of this disclosure.

The loading unit 30 may also be configured for use with other surgical apparatus, such as robotic devices/instruments. For a detailed description of the structure and function of exemplary robotic systems, reference may be made to U.S. Pat. Nos. 6,231,565 and 8,828,023, the entire contents of each of which are incorporated herein by reference.

The loading unit 30 includes a tool or jaw assembly 32 including first and second jaw members 32a, 32b. One or both of the first and second jaw members 32a, 32b is pivotable with respect to the other such that the tool assembly 32 is movable between an open position in which the first and second jaw members 32a, 32b are spaced apart with respect to each other, and a closed position in which the first and second jaw members 32a, 32b are substantially adjacent each other.

The loading unit 30 is a disposable loading unit (“DLU”) that is releasably secured to the elongated tubular body 20 and thus, replaceable with a new loading unit 30. The loading unit 30 may be a single use loading unit (“SULU”) that is used one time and then replaced to facilitate multiples uses of the surgical stapling apparatus 1 on a patient. For example, during a surgical procedure, the surgical stapling apparatus 1 can be used to staple and cut tissue, and the entire SULU is replaced after each staple and cut operation of the surgical stapling apparatus 1. The loading unit 30 may be a multi-use loading unit (“MULU”) that is re-useable a predetermined number of times. For example, during a surgical procedure, the surgical stapling apparatus 1 can be used to staple and cut tissue, and a reload assembly (e.g., a staple cartridge 42) of the MULU is replaced after each staple and cut operation of the surgical stapling apparatus 1 a pre-determined number of times before the entire MULU is replaced. Alternatively, the loading unit 30 may be permanently affixed to the elongated tubular body 20.

The first jaw member 32a of the tool assembly 32 includes a staple cartridge assembly 40 and the second jaw member 32b of the tool assembly 32 includes an anvil assembly 50. The staple cartridge assembly 40 includes a staple cartridge 42 that may be removably and/or replaceably attached to a cartridge carrier 44 of the staple cartridge assembly 40. The staple cartridge 42 may be any staple cartridge used in surgical stapling apparatus within the purview of those skilled in the art that includes a plurality of fasteners (e.g., staples) releasably retained therein.

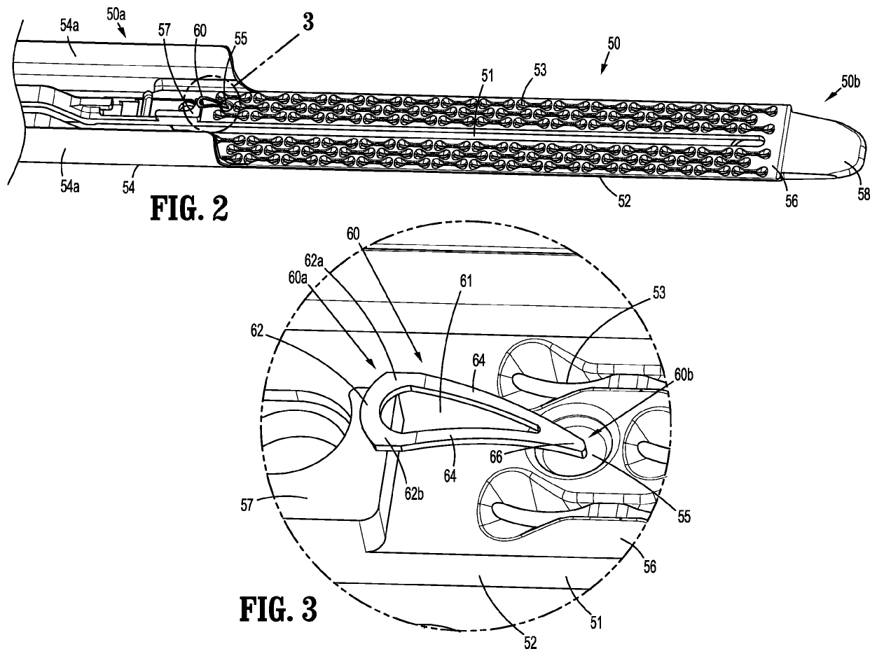

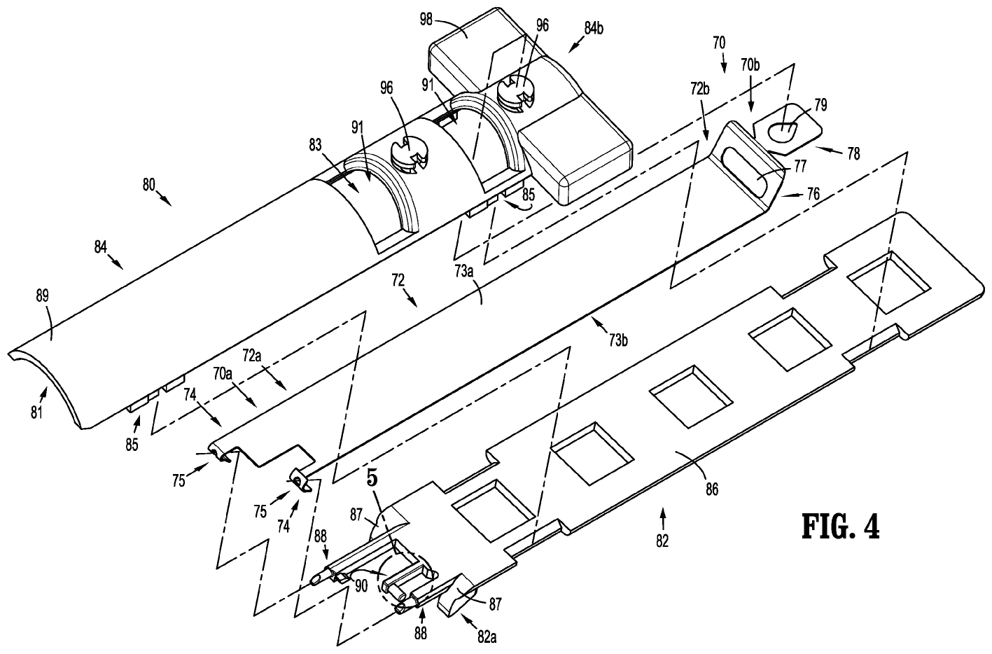

As shown in FIG. 2, the anvil assembly 50 includes an anvil plate 52 and an anvil cover 54 secured over the anvil plate 52. The anvil plate 52 has a central longitudinal slot 51 formed therein and a plurality of staple forming pockets or cavities 53 defined in an inward or tissue facing surface 56 thereof. A notch or indentation 55 is also defined in the tissue facing surface 56 of the anvil plate 52 and a step or protuberance 57 is defined on the tissue facing surface 56 within a proximal end portion 50a of the anvil assembly 50. The step 57 is aligned with, and disposed proximal to and in spaced relation from, the notch 55. The proximal end portion 50a of the anvil assembly 50 also includes a snap clip 60 configured to releasably secure a proximal portion 70a (FIG. 4) of an anvil buttress 70 to the anvil assembly 50. The anvil cover 54 includes side walls 54a extending outwardly towards the staple cartridge assembly 40 (FIG. 1) at the proximal end portion 50a of the anvil assembly 50 on opposed sides of the anvil plate 52. A distal end portion 50b of the anvil assembly 50 includes an anvil tip 58 extending distally of the staple forming pockets 53.

As shown in FIG. 3, the snap clip 60 is disposed laterally of the central longitudinal slot 51 of the anvil plate 52. The snap clip 60 has a proximal end 60a and a distal end 60b. The proximal end 60a of the snap clip 60 is anchored or fixedly secured to the tissue facing surface 56 of the anvil plate 52 proximally of the staple forming pockets 53. Specifically, the proximal end 60a of the snap clip 60 is attached to the step 57 defined on the tissue facing surface 56. In some aspects, the proximal end 60a of the snap clip 60 is welded to the tissue facing surface 56. The distal end 60b of the snap clip 60 is a free end that is movable relative to the tissue facing surface 56 of the anvil plate 52. Specifically, the distal end 60b of the snap clip 60 is movable into and out of the notch 55 defined in the tissue facing surface 56 of the anvil plate 52.

The snap clip 60 has a generally tear-drop shape defining an opening 61 therethrough. The snap clip 60 includes a proximal base 62 at the proximal end 60a of the snap clip 60, which defines the largest width dimension of the snap clip 60, and a distal tip 66 at the distal end 60b of the snap clip 60, which defines the smallest dimension of the snap clip 60. A pair of arms 64 (referred to herein generally as arms) extend from opposed sides 62a, 62b of the proximal base 62, converging towards one another and connecting at the distal tip 66. This configuration renders the snap clip 60 capable of switching between a concave or closed position and a convex or open position in response to the application of an external force thereon. It should be understood, however, that other configurations of the snap clip 60 are envisioned so long as the configuration allows for stable movement between the concave and convex positions. The snap clip 60 may be formed from a metal or plastic capable of providing the biasing force necessary to move the snap clip 60 between the concave and convex positions.

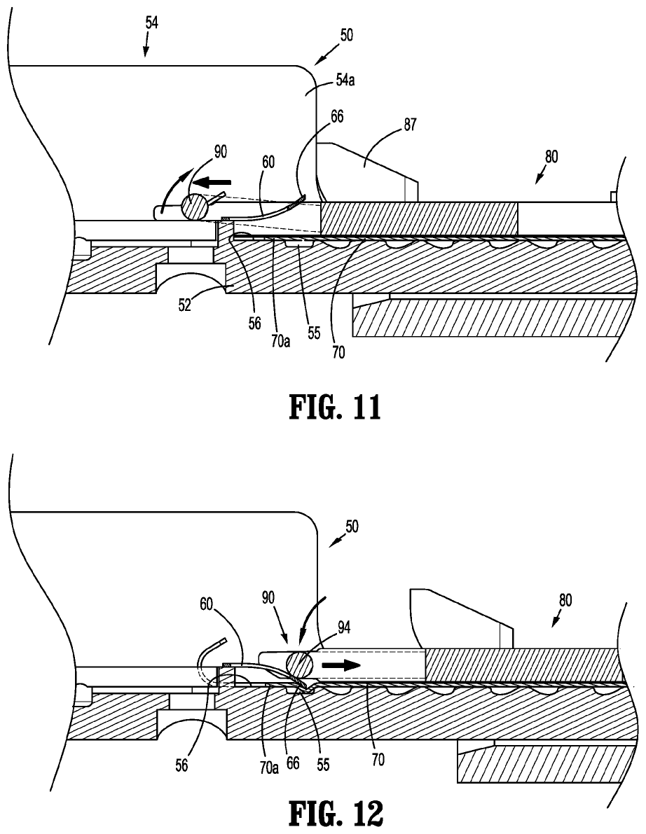

In the concave or closed position, as seen in FIGS. 3, 8, and 9, the snap clip 60 has a generally concave shape relative to the tissue facing surface 56 of the anvil plate 52 with the distal tip 66 of the snap clip 60 positioned against the tissue facing surface 56 (e.g., within the notch 55 defined in the tissue facing surface 56). In the convex or open position, as seen in FIG. 11, the snap clip 60 has a generally convex shape relative to the tissue facing surface 56 of the anvil plate 52 with the distal tip 66 of the snap clip 60 spaced from the tissue facing surface 56. The open position enables the anvil buttress 70 to be positioned between the snap clip 60 and the tissue facing surface 56, and the closed position enables the snap clip 60 to retain the anvil buttress 70 against the tissue facing surface 56 of the anvil plate 52. The snap clip 60 is movable back and forth between the open and closed positions.

Turning now to FIG. 4, an anvil buttress 70 (also referred to herein generally as a surgical buttress) is shown separated from an anvil buttress loading tool 80 (also referred to herein generally as a loading tool). The anvil buttress 70 includes a buttress body 72 having a tissue contacting surface 73a and an anvil contacting surface 73b. The body 72 has a generally rectangular shape that is sized to cover the tissue facing surface 56 (FIG. 2) of the anvil assembly 50. A pair of proximal tabs 74 (also referred to herein generally as proximal tabs) extends proximally from a proximal end 72a of the buttress body 72, with each proximal tab 74 defining a proximal opening 75 therethrough. The proximal openings 75 are sized and shaped to receive proximal protrusions 88 of the anvil buttress loading tool 80 therethrough.

A distal tab 76 extends distally from a distal end 72b of the buttress body 72 and defines a distal window 77 therethrough. The distal window 77 is sized and shaped to receive and frictionally engage the anvil tip 58 (FIG. 2). A distal flap 78 extends distally from the distal tab 76 and defines a distal opening 79 therethrough. The distal opening 79 is sized and shaped to releasably engage a distal protrusion 96 of the anvil buttress loading tool 80. In some aspects, a plurality of perforations, a frangible joint, or the like is disposed between the distal tab 76 and the distal flap 78 to aid in separating the distal flap 78 from the anvil buttress 70 after loading the anvil buttress 70 onto the anvil assembly 50 (FIG. 2). In aspects, the distal tab 76 is bent at an angle relative to the buttress body 72 to aid in securing the distal tab 76 onto the anvil tip 58. In some aspects, the distal tab 76 is stiffened to retain the bend relative to the buttress body 72 and, in other aspects, the distal tab 76 is bent relative to the buttress body 72 by engagement of the distal flap 78 with the distal protrusion 96 of the anvil buttress loading tool 80.

The anvil buttress 70 is fabricated from biocompatible materials which are bioabsorbable or non-absorbable, natural or synthetic materials. It should be understood that a single or combination of natural, synthetic, bioabsorbable, and/or non-bioabsorbable materials may be used to form the anvil buttress 70. In aspects, the anvil buttress 70 is formed from a single sheet of material that is cut to shape. In other aspects, the anvil buttress 70 is formed from a plurality of sheets of material, that are fabricated from the same or different materials, and/or the components (e.g., the buttress body, the tabs, the flap, etc.) of the anvil buttress 70 are formed from the same or different materials that are attached to one another by, for example, welding, using adhesive, tying sutures, etc.

The anvil buttress 70 may be porous, non-porous, or combinations thereof. Suitable porous structures include, for example, fibrous structures (e.g., knitted structures, woven structures, and non-woven structures) and/or foams (e.g., open or closed cell foams). Suitable non-porous structures include, for example, films. The anvil buttress 70 may be a single porous or non-porous layer, or include a plurality of layers including any combination of porous and non-porous layers. For example, the anvil buttress may include multiple porous and non-porous layers that are stacked in an alternating manner. In another example, the anvil buttress may be formed in a “sandwich-like” manner wherein the outer layers are porous and the inner layer(s) are non-porous, or vice versa.

Porous layer(s) in a surgical buttress may enhance the ability of the surgical buttress to absorb fluid, reduce bleeding, and/or seal a wound. Also, the porous layer(s) may allow for tissue ingrowth to fix the surgical buttress in place. Non-porous layer(s) in a surgical buttress may enhance the ability of the surgical buttress to resist tears and perforations during the manufacturing, shipping, handling, and/or stapling processes. Also, non-porous layer(s) may retard or prevent tissue ingrowth from surrounding tissues thereby acting as an adhesion barrier and preventing the formation of unwanted scar tissue.

With continued reference to FIG. 4, the anvil buttress loading tool 80 is configured to releasably retain the anvil buttress 70 therein and to receive the anvil assembly 50 (FIG. 2) therein for loading the anvil buttress 70 onto the anvil assembly 50 (e.g., transferring the anvil buttress 70 from the anvil buttress loading tool 80 to the anvil assembly 50). The anvil buttress loading tool 80 has a two-part construction including a carrier 82 and a cover 84. The carrier 82 and the cover 84 are releasably engaged with each other via snap fit connectors 85, however, other mating structures and relationships are contemplated to releasably secure the carrier 82 and the cover 84 together. Alternatively, the carrier 82 and the cover 84 may be formed as a single, non-separable piece.

Together, the carrier 82 and the cover 84 define a proximal opening 81 into a cavity 83 defined between the carrier 82 and the cover 84. The proximal opening 81 and the cavity 83 correspond in size and shape with the anvil assembly 50 (FIG. 2) and thus, are configured to slidably receive the anvil assembly 50 in an orientation in which the tissue facing surface 56 (FIG. 2) of the anvil plate 52 faces the carrier 82.

The carrier 82 has a support plate 86 dimensioned to accommodate the buttress body 72 of the anvil buttress 70 thereon. A proximal end portion 82a of the carrier 82 includes a pair of stop members 87 (referred to herein generally as stop members), a pair of proximal protrusions 88 (referred to herein generally as proximal protrusions), and a clip roller 90. The stop members 87 extend proximally from opposed sides of the support plate 86 and are configured and dimensioned to contact the side walls 54a (FIG. 2) of the anvil cover 54 when the anvil buttress loading tool 80 is fully loaded onto the anvil assembly 50. The proximal protrusions 88 extend proximally from the support plate 86 and are disposed between the stop members 87. The proximal protrusions 88 are configured and dimensioned to engage the proximal tabs 74 of the anvil buttress 70 to retain a proximal portion 70a of the anvil buttress 70 on the anvil buttress loading tool 80. The clip roller 90 extends proximally from the support plate 86 and is disposed between the proximal protrusions 88.

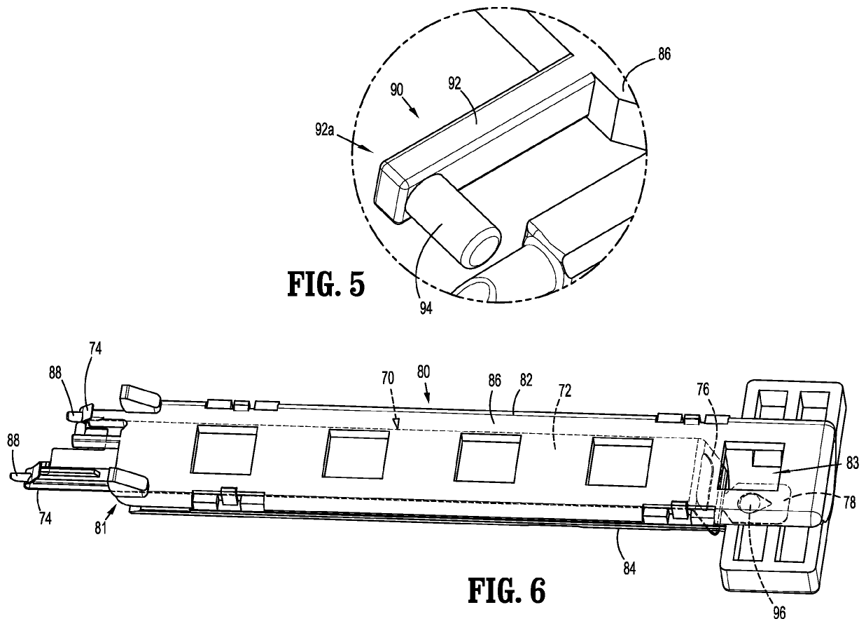

As shown in FIG. 5, the clip roller 90 includes a post 92 extending proximally from the support plate 86 and a cylinder 94 extending laterally from a proximal end portion 92a of the post 92. The position of the cylinder 94 corresponds with the position of the snap clip 60 on the anvil assembly 50. The cylinder 94 is sized and shaped to apply a force onto the snap clip 60 (e.g., acts as a camming surface) when the anvil buttress loading tool 80 is moved onto and off of the anvil assembly 40 to change the configuration of the snap clip 60 between the open and closed positions. In some aspects, the cylinder 94 rotates relative to the post 92 and, in other aspects, the cylinder 94 is non-rotatably connected to the post 92.

With reference again to FIG. 4, the cover 84 is sized and shaped to cover the support plate 86 of the carrier 82. The proximal end portion 82a of the carrier 82 (e.g., the stop members 87, the proximal protrusions 88, and the clip roller 90) is not covered by the cover 84. A distal end portion 84b of the cover 84 includes at least one distal protrusion 96 extending outwardly from an outer surface 89 of the cover 84 and at least one distal opening 91 defined through the cover 84 and disposed proximal to the distal protrusion 96. The distal opening 91 is configured and dimensioned for passage of the distal flap 78 out of the cavity 83 of the anvil buttress loading tool 80, and the distal protrusion 96 is configured and dimensioned to engage the distal flap 78 of the anvil buttress 70 to retain a distal portion 70b of the anvil buttress 70 on the anvil buttress loading tool 80. In some aspects, as seen in FIG. 4 for example, the cover 84 includes a plurality of distal openings and protrusions 91, 96 (e.g., two distal openings 91 and two distal protrusions 96) to accommodate anvil buttresses 70 of different lengths. The cover 84 also includes a handle 98 extending laterally from opposed sides of the distal end portion 84b of the cover 84 for ease in handling by a user.

In a method of loading the anvil buttress loading tool 80 with the anvil buttress 70, the cover 84 of the anvil buttress loading tool 80 is separated from the carrier 82, as seen in FIG. 4, so that the anvil buttress 70 may be placed into the anvil buttress loading tool 80. The anvil buttress 70 is positioned on the carrier 82 with the buttress body 72 positioned against the support plate 86 and the proximal tabs 74 engaged with the proximal protrusions 88. The distal flap 78 is positioned through the distal opening 91 in the cover 84 and the distal opening 79 in the distal flap 78 is engaged with the distal protrusion 96 on the cover 84. The cover 84 is then snap fitted onto the carrier 82, as shown in FIG. 6, to load the anvil buttress 70 in the anvil buttress loading tool 80. In the loaded configuration, the buttress body 72 and the distal tab 76 are retained within the cavity 83 of the anvil buttress loading tool 80, the distal flap 78 extends distally out of the distal opening 91, and the proximal tabs 74 extend proximally out of the proximal opening 81. The proximal tabs 74 and the distal flap 78 help maintain alignment of the anvil buttress 70 on the anvil buttress loading tool 80. Alternatively, the anvil buttress 70 may be passed into (e.g., slid into) the cavity 83 of the anvil buttress loading tool 80 through the proximal opening 81, and the proximal tabs 74 and the distal flap 78 are manipulated to engage the respective proximal and distal protrusions 88, 96.

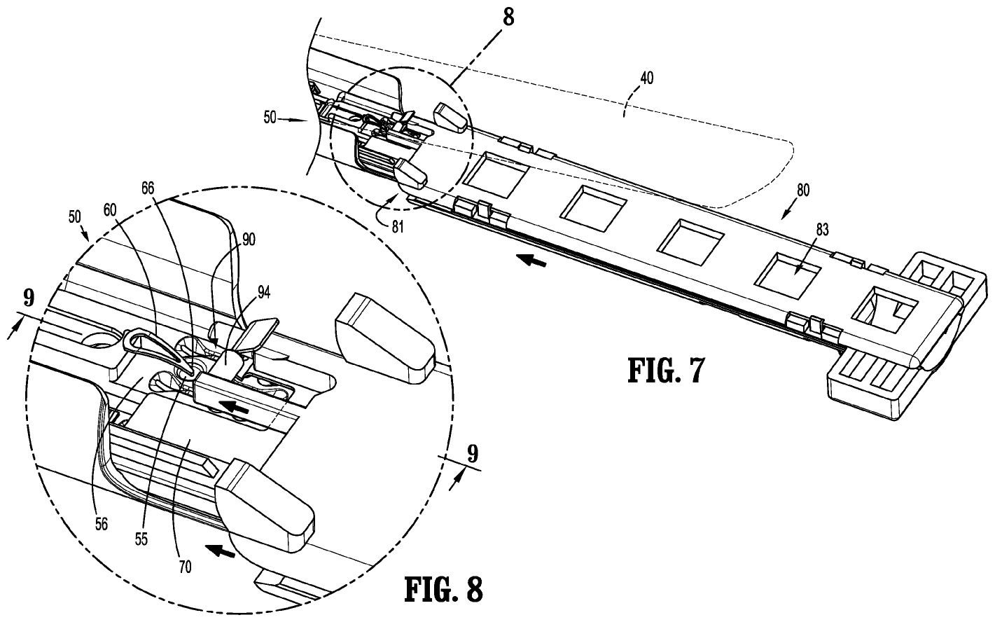

In a method of loading the anvil assembly 50 with the anvil buttress 70, the anvil buttress loading tool 80, loaded with the anvil buttress 70, as seen in FIG. 6, is positioned relative to the anvil assembly 50, seen in FIG. 2, with the proximal opening 81 into the cavity 83 of the anvil buttress loading tool 80 aligned with the anvil tip 58 of the anvil assembly 50, and with the anvil buttress 70 facing the tissue facing surface 56 of the anvil assembly 50. As shown in FIGS. 7 and 8, the anvil buttress loading tool 80 is slid proximally over the anvil assembly 50 to advance the anvil buttress loading tool 80 onto the anvil assembly 50 such that the tissue facing surface 56 of the anvil assembly 50 is adjacent to or in contact with the anvil buttress 70. Additionally, or alternatively, the anvil assembly 50 may be slid distally into the cavity 83 of the anvil buttress loading tool 80 through the proximal opening 81.

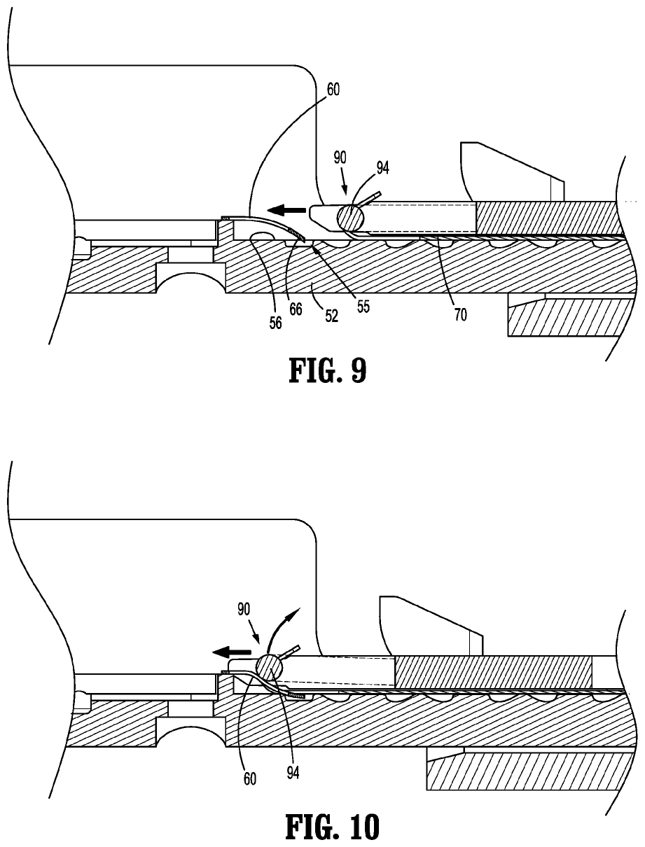

During this sliding movement, as shown in FIGS. 8 and 9, the clip roller 90 is distal to and aligned with the snap clip 60, which is disposed in the closed position. As the clip roller 90 approaches the snap clip 60, the cylinder 94 of the clip roller 90 contacts the snap clip 60, as shown in FIG. 10, effectively snapping the snap clip 60 from the closed position to the open position, as shown in FIG. 11, as the clip roller 90 moves proximally thereover and beyond the snap clip 60. In the open position, the distal tip 66 of the snap clip 60 is raised off of the tissue facing surface 56 (e.g., out of the notch 55) of the anvil assembly 50, allowing the proximal portion 70a of the anvil buttress 70 to slid between the tissue facing surface 56 and the snap clip 60. The anvil buttress loading tool 80 is advanced onto the anvil assembly 50 until the stop members 87 contact the side walls 54a of the anvil cover 54, as seen in FIG. 11. When the anvil assembly 50 is fully advanced into the anvil buttress loading tool 80, the anvil tip 58 (FIG. 2) extends through the distal window 77 (FIG. 4) defined through the anvil buttress 70.

After the anvil assembly 50 is fully advanced into the anvil buttress loading tool 80, the anvil buttress loading tool 80 may be separated from the anvil assembly 50 by sliding the anvil buttress loading tool 80 and/or the anvil assembly 50 in opposite directions. As the anvil assembly 50 and the anvil buttress loading tool 80 are separated from each other, the cylinder 94 of the clip roller 90 passes back over the snap clip 60, as shown in FIG. 12, moving the snap clip 60 back to the closed position. The distal tip 66 of the snap clip 60 snaps back down towards the tissue facing surface 56 of the anvil assembly, and more specifically into the notch 55, thereby capturing the proximal end portion 70a of the anvil buttress 70 to the anvil assembly 50. Additionally, the distal flap 78 (FIG. 6) tears away from the anvil buttress 70 and is retained on the anvil buttress loading tool 80 during separation thereof from the anvil assembly 50. Alternatively, the distal flap 78 may rip or otherwise detach from the distal protrusion 96 during separation of the anvil buttress loading tool 80 from the anvil assembly 50 and remain attached to the anvil buttress 70.

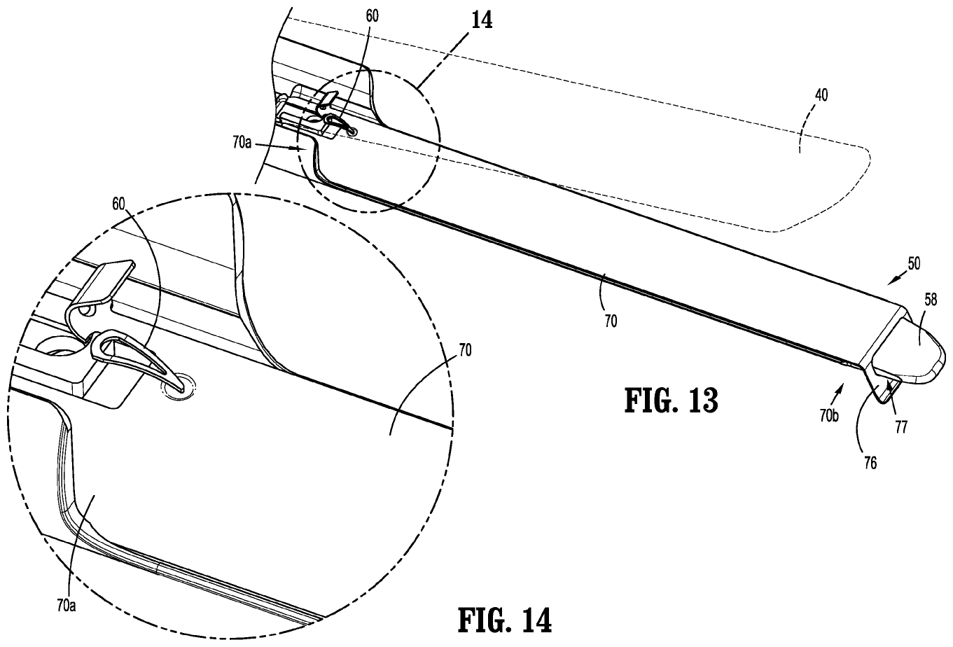

After the anvil buttress loading tool 80 is separated from the anvil assembly 50, as shown in FIGS. 13 and 14, the anvil assembly 50 is loaded with the anvil buttress 70. Specifically, the proximal end portion 70a of the anvil buttress 70 is retained on the anvil assembly 50 by the snap clip 60 and the distal end portion 70b of the anvil buttress 70 is retained on the anvil assembly 50 by engagement of the distal tab 76 with the anvil tip 58 through the distal window 77.

The surgical stapling apparatus 1 (FIG. 1), with the anvil assembly 50 loaded with the anvil buttress 70, is ready for use. In aspects, the staple cartridge assembly 40 is pre-loaded and/or loaded with a cartridge buttress (not explicitly shown). The cartridge buttress may be releasably secured to the staple cartridge assembly 40 via any suitable attachment feature within the purview of those skilled in the art, such as, for example, mechanical attachment features (e.g., sutures, pins), chemical attachment features (e.g., adhesive), and/or attachment methods (e.g., welding).

In operation, with the tool assembly 32 loaded with the anvil buttress 70, as described above, the surgical stapling apparatus 1 is used in accordance with methods known by those skilled in the art. Once the staple cartridge and anvil assemblies 40, 50 are clamped onto tissue, the surgical stapling apparatus 1 is fired, thereby stapling the anvil buttress 70 to the tissue, as well as cutting and dividing the tissue and the anvil buttress 70 disposed between the rows of formed staples. When firing is complete and the staple cartridge and anvil assemblies 40, 50 are unclamped, the anvil buttress 70, which is now stapled to the tissue, pulls away from the anvil assembly 50, and the tool assembly 32 can be removed from the surgical site. The snap clip 60 remains engaged with the anvil buttress 70 throughout the clamping and firing process and remains in the closed position following firing, but the anvil buttress 70, now stapled into the tissue, can slide out from the snap clip 60. In some aspects, the used staple cartridge 42 may be removed from the tool assembly 34 and replaced with a new staple cartridge 42 and, in some other aspects, the loading unit 30 may be replaced with a new loading unit 30. A new anvil buttress 70 may be installed onto the anvil assembly 50 by an anvil buttress loading tool 80, as needed or desired, as described above.

It should be understood that the tissue facing surface 56 of the anvil plate 52 and/or the snap clip 60 may have other configurations. For example, while the notch 55 is defined between a proximal-most pair of staple forming pockets 53, as seen in FIG. 3, it should be understood that the notch 55 may be defined proximal of all the staple forming pockets 53 or no notch may be provided and the distal end 60b of the snap clip 60 may rest directly against the tissue facing surface 56 when the snap clip 60 is in the closed position. As another example, the step 57 may be omitted and the proximal end 60a of the snap clip 60 may be attached directly to the tissue facing surface 56 of the anvil plate 52.

As yet another example, a second snap clip 60 may be disposed on the other side of the central longitudinal slot 51 of the tissue facing surface 56. In such aspects, the configuration of the anvil plate 52 and the anvil buttress loading tool 80 on each side of the central longitudinal slot 51 are mirror images of each other. Alternatively, a different anvil buttress retention mechanism may be utilized on the other side central longitudinal slot 51, and the anvil buttress loading tool 80 may be modified to actuate the anvil buttress retention mechanism.

While aspects of the disclosure have been shown in the drawings, it is not intended that the disclosure be limited thereto, as it is intended that the disclosure be as broad in scope as the art will allow and that the specification be read likewise. It is to be understood, therefore, that the disclosure is not limited to the precise aspects described, and that various other changes and modifications may be effected by one skilled in the art without departing from the scope or spirit of the disclosure. Therefore, the above description should not be construed as limiting, but merely as exemplifications of aspects of the disclosure. Thus, the scope of the disclosure should be determined by the appended claims and their legal equivalents, rather than by the examples given.

What is claimed is:

1. An anvil buttress loading system comprising:
an anvil assembly including a tissue facing surface and a snap clip disposed on the tissue facing surface, the snap clip movable between a closed position having a generally concave shape relative to the tissue facing surface and an open position having a generally convex shape relative to the tissue facing surface;
an anvil buttress loading tool including a carrier having a support plate and a clip roller extending proximally from the support plate; and
an anvil buttress including a buttress body,
the anvil buttress releasably retained on the anvil buttress loading tool with the buttress body positioned on the support plate, the anvil buttress transferrable from the anvil buttress loading tool to the anvil assembly such that the buttress body is captured between the snap clip and the tissue facing surface of the anvil assembly, the snap clip movable from the closed position to the open position by the clip roller when advancing the anvil buttress loading tool onto the anvil assembly and from the open position to the closed position when separating the anvil buttress loading tool from the anvil assembly.

2. The anvil buttress loading system according to claim 1, wherein the clip roller is configured to move a free end of the snap clip off of the tissue facing surface of the anvil assembly when the anvil buttress loading tool is advanced onto the anvil assembly, and to move the free end of the snap clip towards the tissue facing surface when the anvil buttress loading tool is separated from the anvil assembly.

3. The anvil buttress loading system according to claim 1, wherein the clip roller includes a post extending proximally from the support plate and a cylinder extending laterally from the post.

4. The anvil buttress loading system according to claim 1, wherein, in the closed position, a free end of the snap clip is adjacent to the tissue facing surface and, in the open position, the free end of the snap clip is spaced from the tissue facing surface.

5. The anvil buttress loading system according to claim 1, wherein the snap clip includes a proximal end secured to the tissue facing surface of the anvil plate and a distal end movable relative to the tissue facing surface, the distal end configured to hold the anvil buttress against the tissue facing surface of the anvil assembly.

6. The anvil buttress loading system according to claim 1, wherein the snap clip has a generally tear drop shape and defines an opening therethrough.

7. The anvil buttress loading system according to claim 6, wherein the snap clip includes a proximal base and a distal tip, the proximal base defining the largest width dimension of the snap clip and the distal tip defining the smallest width dimension of the snap clip.

8. The anvil buttress loading system according to claim 7, wherein a pair of arms extend from opposed sides of the proximal base, the pair of arms converging towards one another and connecting at the distal tip.

9. The anvil buttress loading system according to claim 1, wherein the snap clip is disposed on a proximal portion of the anvil assembly and is configured to retain a proximal portion of the anvil buttress against the tissue facing surface of the anvil assembly.

10. The anvil buttress loading system according to claim 9, wherein the anvil assembly includes an anvil tip and the anvil buttress includes a distal window defined through a distal portion of the anvil buttress, the distal window configured to engage the anvil tip to retain the distal portion of the anvil buttress on the anvil assembly.

11. An anvil buttress loading assembly comprising:
an anvil buttress loading tool including a carrier having a support plate and a clip roller extending proximally from the support plate; and
an anvil buttress including a buttress body,
the anvil buttress releasably retained on the anvil buttress loading tool with the buttress body positioned on the support plate.

12. The anvil buttress loading assembly according to claim 11, wherein the clip roller includes a post extending proximally from the support plate and a cylinder extending laterally from the post.

13. The anvil buttress loading assembly according to claim 11, wherein the anvil buttress loading tool includes a pair of proximal protrusions extending from the support plate and the anvil buttress includes a pair of proximal tabs extending from the buttress body, the pair of proximal tabs engaged with the pair of proximal protrusions.

14. The anvil buttress loading assembly according to claim 13, wherein the clip roller is disposed between the pair of proximal protrusions.

15. The anvil buttress loading assembly according to claim 11, wherein the anvil buttress loading tool further includes a cover disposed over the support plate of the carrier.

16. The anvil buttress loading assembly according to claim 15, wherein the cover includes a distal protrusion extending outwardly from an outer surface of the cover and a distal opening disposed proximal to the distal protrusion, and the anvil buttress includes a distal flap, the distal flap extending through the distal opening and engaged with the distal protrusion.

17. A tool assembly comprising:
a staple cartridge assembly; and
an anvil assembly including a tissue facing surface and a snap clip disposed on the tissue facing surface, the snap clip movable between a closed position having a generally concave shape relative to the tissue facing surface and an open position having a generally convex shape relative to the tissue facing surface.

18. The tool assembly according to claim 17, wherein, in the closed position, a free end of the snap clip is adjacent to the tissue facing surface and, in the open position, the free end of the snap clip is spaced from the tissue facing surface.

19. The tool assembly according to claim 17, further including an anvil buttress having a proximal portion and a distal portion, the proximal portion releasably retained on the anvil assembly by the snap clip when the snap clip is in the closed position.

20. The tool assembly according to claim 19, wherein the anvil assembly includes an anvil tip and the distal portion of the anvil buttress defines a distal window therethrough, the distal portion releasably retained on the anvil assembly by engagement of the anvil tip through the distal window.
